(19) United States
(12) Patent Application Publication
(10) Pub. No.: US 2021/0052492 A1
(43) Pub. Date: Feb. 25, 2021

(54) Cannabinoid Dosing Regime For Psoriasis

(71) Applicant: Botanix Pharmaceuticals Ltd., North Peth, Western Autralia (AU)

(72) Inventors: Matthew Callahan, Pennsylvania, PA (US); Michael Thurn, Kangaroobie, New South Wales (AU)

(21) Appl. No.: 16/964,787

(22) PCT Filed: Jan. 24, 2019
(86) PCT No.: PCT/AU2019/050052

§ 371 Date: Jul. 24, 2020

Related U.S. Application Data
(60) Provisional application No. 62/621,244, filed on Jan. 24, 2018.
(60) Provisional application No. 62/736,044, filed on Sep. 25, 2018.

(30) Foreign Application Priority Data
Jan. 24, 2018 (AU) 2018900225
Sep. 25, 2018 (AU) 2018903598

(51) Int. Cl. A61K 9/06; A61K 9/00; A61K 31/05; A61K 31/352

(52) U.S. Cl. CPC A61K 9/06; A61K 31/352; A61K 31/05; A61K 9/0014

(57) ABSTRACT

A treatment regime for use in the treatment or prevention of psoriasis, said regime comprising the administration of: a) between 50 mg and 3000 mg of a topical liquid or gel composition comprising between 1% w/w and 15% w/w cannabinoid, wherein the cannabinoid is dissolved in the liquid or gel composition.

### TECHNICAL FIELD

A topical dosing regimen for the treatment or prevention of psoriasis using cannabinoids.

### BACKGROUND ART

Most mammalian skin, including human skin, comprises three layers: (i) an epidermis layer; (ii) a dermis layer; and (iii) a hypodermis layer. The epidermis itself is made up of two layers, the outer stratum corneum and the inner epidermal basal layer.

The majority of skin conditions involve inflammation triggered by some insult to the skin. Keratinocytes respond quickly to environmental stimuli (e.g., UV radiation (UVR), allergens, irritants or physical damage) by producing a variety of inflammatory mediators, including cytokines (e.g., IL-I, TNF-alpha, and IL-6) and chemokines (e.g., IL-8). One of the most active inflammatory mediators is PGE-2 (Prostaglandin E2) and, of course, many topical dermatology drugs have been designed to lower levels of PGE-2. The fibroblasts in the dermis also produce PGE-2 along with a variety of chemokines, cytokines and matrix destroying enzymes such as collagenase (MMP-I).

Psoriasis is a long-lasting autoimmune disease which is characterized by patches of abnormal skin. These skin patches are typically red, itchy, and scaly. They may vary in severity from small and localized to complete body coverage. Injury to the skin can trigger psoriatic skin changes at that spot, which is known as Koebner phenomenon.

There are five main types of psoriasis: plaque, guttate, inverse, pustular, and erythrodermic. Plaque psoriasis, also known as psoriasis vulgaris, makes up about 90% of cases. It typically presents with red patches with white scales on top. Areas of the body most commonly affected are the back of the forearms, shins, around the navel, and the scalp. Guttate psoriasis has drop-shaped lesions. Pustular psoriasis presents with small non-infectious pus-filled blisters. Inverse psoriasis forms red patches in skin folds. Erythrodermic psoriasis occurs when the rash becomes very widespread, and can develop from any of the other types. Fingernails and toenails are affected in most people at some point in time. This may include pits in the nails or changes in nail colour.

Psoriasis is generally thought to be a genetic disease which is triggered by environmental factors. Symptoms often worsen during winter and with certain medications such as beta blockers or NSAIDs, and infections and psychological stress may also play a role. The underlying mechanism involves the immune system reacting to skin cells.

Pustular psoriasis appears as raised bumps filled with non-infectious pus (pustules). The skin under and surrounding the pustule is red and tender. Pustular psoriasis can be localized, commonly to the hands and feet (palmoplantar pustulosis), or generalized with widespread patches occurring randomly on any part of the body. Acrodermatitis continua is a form of localized psoriasis limited to the fingers and toes that may spread to the hands and feet. Pustulosis palmaris et plantaris is another form of localized pustular psoriasis similar to acrodermatitis continua with pustules erupting from red, tender, scaly skin found on the palms of the hands and the soles of the feet.

Generalized pustular psoriasis (pustular psoriasis of von Zumbusch), also known as impetigo herpetiformis during pregnancy, is a rare and severe form of psoriasis that may require hospitalization. The development of generalized pustular psoriasis is often caused by an infection, abrupt withdrawal of topical corticosteroid treatment, pregnancy, hypocalcemia, medications, or following an irritating topical treatment for plaque psoriasis. This form of psoriasis is characterized by an acute onset of numerous pustules on top of tender red skin. This skin eruption is often accompanied by a fever, muscle aches, nausea, and an elevated white blood cell count.

Annular pustular psoriasis (APP), a rare form of generalized pustular psoriasis, is the most common type seen during childhood. APP tends to occur in women more frequently than in men, and is usually less severe than other forms of generalized pustular psoriasis such as impetigo herpetiformis. This form of psoriasis is characterized by ring-shaped plaques with pustules around the edges and yellow crusting. APP most often affects the torso, neck, arms, and legs.

Additional types of psoriasis affecting the skin include inverse psoriasis, guttate psoriasis, oral psoriasis, and seborrheic-like psoriasis.

Inverse psoriasis (also known as flexural psoriasis) appears as smooth, inflamed patches of skin. The patches frequently affect skin folds, particularly around the genitals (between the thigh and groin), the armpits, in the skin folds of an overweight abdomen (known as panniculus), between the buttocks in the intergluteal cleft, and under the breasts in the inframammary fold. Heat, trauma, and infection are thought to play a role in the development of this atypical form of psoriasis.

Napkin psoriasis is a subtype of psoriasis common in infants characterized by red papules with silver scale in the diaper area that may extend to the torso or limbs. Napkin psoriasis is often misdiagnosed as napkin dermatitis (diaper rash).

Guttate psoriasis is characterized by numerous small, scaly, red or pink, droplet-like lesions (papules). These numerous spots of psoriasis appear over large areas of the body, primarily the trunk, but also the limbs and scalp. Guttate psoriasis is often triggered by a streptococcal infection, typically streptococcal pharyngitis.

Oral psoriasis is very rare, in contrast to lichen planus, another common papulosquamous disorder that commonly involves both the skin and mouth. When psoriasis involves the oral mucosa (the lining of the mouth), it may be asymptomatic, but it may appear as white or grey-yellow plaques. Fissured tongue is the most common finding in those with oral psoriasis and has been reported to occur in 6.5-20% of people with psoriasis affecting the skin.

Seborrheic-like psoriasis is a common form of psoriasis with clinical aspects of psoriasis and seborrheic dermatitis, and may be difficult to distinguish from the latter. This form of psoriasis typically manifests as red plaques with greasy scales in areas of higher sebum production such as the scalp, forehead, skin folds next to the nose, skin surrounding the mouth, skin on the chest above the sternum, and in skin folds.

Cannabinoids have been proposed as a treatment for skin conditions such as acne. However, the amount of active agent in the available topical creams is usually very low, and there is little evidence that a therapeutically useful dose is being provided to the user.

It is against this background that the present invention has been developed. The present invention seeks to provide a high dosage composition of cannabinoids for topical use to treat or prevent psoriasis, or to provide the consumer with a useful therapeutic or commercial choice.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

In accordance with the present invention, there is provided a regime for use in the treatment or prevention of psoriasis, said regime comprising the administration of:
a) between 50 mg and 3000 mg of a topical composition comprising between 1% w/w and 15% w/w cannabinoid to the skin of a subject in need of such treatment or prevention.

Preferably, the composition comprises between 2% w/w and 25% w/w cannabinoid, more preferably 5% w/w, 10% w/w or 20% w/w.

Preferably, the composition of the treatment regime is administered to the skin between 1 and 5 times per day, more preferably once or twice per day.

Preferably, the composition of the treatment regime delivers between 20 mg and 400 mg of cannabinoid per administration, more preferably, 27.5 mg, 37.5 mg or 75 mg of cannabinoid per administration.

Preferably, the total daily dose applied to the skin is between 20 mg and 2000 mg cannabinoid, more preferably 27.5 mg, 55 mg, 75 mg or 110 mg.

The present invention further provides a method for treating or preventing psoriasis, said method comprising the administration of:
a) between 50 mg and 3000 mg of a topical composition comprising between 1% w/w and 15% w/w cannabinoid to the skin of a subject in need of such treatment or prevention.

Preferably, the composition of the treatment regime is in a liquid or gel form.

Preferably, composition of the treatment regime delivers the cannabinoid in a composition comprising: (i) a volatile solvent; and (ii) a residual solvent that is less volatile than (i).

Preferably the volatile solvent is a non-polymeric siloxane, a C₂-C₆ alcohol or a mixture of both. Preferably the composition comprises: 85-95% w/w siloxane and 1-10% wt/wt C₂-C₆ alcohol; 85-95% w/w non-polymeric siloxane; or 70-80% w/w wt/wt C₂-C₆ alcohol.

Preferably the residual solvent is a compound from the list comprising: fatty acids, fatty acid alcohols, fatty alcohols, glycols, alkanes, ethers of any of these, and combinations thereof. Preferably the composition comprises 1-10% wt/wt of residual solvent.

The present invention further provides for the use of between 50 mg and 3000 mg of a topical composition comprising between 1% w/w and 15% w/w cannabinoid for the treatment or prevention of psoriasis in a subject in need of such treatment or prevention.

The present invention further provides for the use of between 1% w/w and 15% w/w cannabinoid for the manufacture of a topical composition for the treatment or prevention of psoriasis, wherein between 50 mg and 3000 mg of the topical composition is administered to the skin of a subject in need of such treatment or prevention.

The present invention further provides for the manufacture of a topical composition comprising between 1% w/w and 15% w/w cannabinoid for use in the treatment or prevention of psoriasis, wherein between 50 mg and 3000 mg of the topical composition is administered to the skin of a subject in need of such treatment or prevention.

The present invention further provides a topical composition comprising between 1% w/w and 15% w/w cannabinoid for use in the treatment or prevention of psoriasis, wherein between 50 mg and 3000 mg of the topical composition is administered to the skin of a subject in need of such treatment or prevention.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a photograph of the psoriasis plaque test (PPT).

FIG. 2 is a diagram of two possible assignment of test field scenarios. 2A is a one plaque test, 2B is a two plaque test.

### DESCRIPTION OF INVENTION

### Detailed Description of the Invention

The present invention is based on the finding that the amount of cannabinoids in the available topical creams for psoriasis treatment is usually very low, and there is little evidence that a therapeutically useful dose is being provided to the user. The average topical cannabinoid cream is labelled to contain between about 300 mg and 750 mg of cannabinoid per 120 mL jar of cream, which if the labelling is correct, provides an average dose, once applied to the skin, of about 5 mg to 15 mg per dose.

The term cannabinoid includes compounds which interact with the cannabinoid receptor and various cannabinoid mimetics, such as certain tetrahydropyran analogs (e.g., Δ⁹-tetrahydrocannabinol, Δ⁸-tetrahydro-cannabinol, 6,6,9-trimethyl-3-pentyl-6H-dibenzo [b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ6-tetrahydrocannabinol-1,1-dimethylheptyl, (+)-(3S,4S)-7-hydroxy-Δ6-tetrahydrocannabinol-1,1-dimethylheptyl, 11-hydroxy-Δ⁹-tetrahydrocannabinol, and Δ8-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-methyl-4-phenylbutoxy]-1,9-phenanthridinediol-1-acetate)); certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenyl-methanone); and certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,2′,3′,4′,5′,6′-hexahydrobiphenyl).

Cannabidiol, as used herein, refers to 2-[3-methyl-6-(1-methylethenyl)-2-cyclohexen-1-yl]-5-pentyl-1,3-benzenediol. The synthesis of cannabidiol is described, for example, in Petilka et al., Helv. Chim. Acta, 52: 1102 (1969) and in Mechoulam et al., J. Am. Chem. Soc., 87:3273 (1965), which are hereby incorporated by reference

Identification of the main cannabinoid receptors (CB1 and CB2), their endogenous lipid ligands (endocannabinoids), biosynthetic pathways and metabolizing enzymes (collectively termed the ECS), coupled with the discovery and/or rational design of numerous exogenous ligands for CB receptors, has triggered an exponential growth in studies exploring the continuously growing regulatory functions of this newly discovered physiological system both in health and disease.

The most extensively studied endocannabinoids are anandamide (N arachidonoylethanolamine, AEA) and 2-arachidonoylglycerol (2-AG). Multiple pathways are involved in synthesis and cellular uptake of these lipid mediators. The most common degradation pathways for AEA and 2-AG are the fatty acid amid hydrolase (FAAH) and monoacylglycerol lipase (MAGL) enzyme. Endocannabinoids, similar to Δ⁹-tetrahydrocannabinol (THC; the main active ingredient of the plant Cannabis sativa), predominantly exert their physiological effects via two main G-protein-coupled cannabinoid receptors; however, numerous additional signalling mechanisms and receptor systems (e.g. transient receptor potential cation channel, subfamily V, member 1; TRPV1) might also be involved. Initially, the CB1-mediated effects were described centrally and CB1 receptors were thought to be restricted to the central nervous system, whereas CB2 was first identified at the periphery in immune cells.

It is considered that CBD may:
inhibit hyperproliferation of keratinocytes;
exert universal anti-inflammatory actions such as:
   decrease primed T-cell activity and also inhibit subsequent B-cell response;
   suppress multiple T-cell populations and inhibit general T-cell activation;
   decrease concentrations of pro-inflammatory mediators and also increase the release of anti-inflammatory cytokines;
   inhibit the effects of IFN-γ and/or decrease IFN-γ levels;
   inhibit the migration, proliferation and cell maturation processes involved in Th17, Th1, and Th2 immune responses; and
have direct antioxidant effects

Without being held to any theory, we believe that the mode of action of CBD for psoriasis involves the suppression of mediators of inflammatory responses. There is a physiological regulatory function of the endocannabinoid system (ECS) in proliferation, differentiation, apoptosis and cytokine, mediator and hormone production of various cell types of the skin and appendages (e.g. hair follicle, sebaceous gland).

In vitro studies have shown CBD to stimulate the human vanilloid receptor type 1 (VR1) and to inhibit anandamide (an endogenous CBD neurotransmitter). These findings have suggested a mode of action for the anti-inflammatory properties of CBD. In vivo studies with intravenous administration of CBD in sensitized guinea-pigs reduced airway obstruction, indicating a potential role of CBD in reducing immune-induced inflammatory reactions. Similarly, CBD injected into rats attenuated cardiac inflammation.

### Treatment Regime

Unless the context requires otherwise, the term ‘psoriasis’, as used herein, means one or more of: pustular psoriasis (including palmoplantar pustulosis, acrodermatitis continua, pustulosis palmaris et plantaris, pustular psoriasis of von Zumbusch, annular pustular psoriasis), inverse psoriasis, napkin psoriasis, guttate psoriasis, oral psoriasis, and seborrheic-like psoriasis.

It has been found that the inflammatory markers (cytokines) produced by skin and immune cells that are required for the development of an inflammatory response, such as in psoriasis. The present invention comprises active agents, in the form of cannabinoids, that suppress the production of a variety of inflammatory responses in cultured skin cells (keratinocytes and fibroblasts), and immune cells (monocytes and T-lymphocytes) and in intact living skin. As a result of blocking these inflammatory processes in the skin, the present compounds in the form of cannabinoids are able to effectively reduce or eliminate a variety of inflammatory symptoms that occur with common skin problem (see Kupczyk et al (2009) Cannabinoid system in the skin—a possible target for future therapies in dermatology Exp Dermatol. 18(8):669-79

High concentrations of dissolved cannabinoids, including cannabidiol (as opposed to solid cannabinoids) are expected to be advantageous in terms of enhancing the relevant extent of delivery into the skin, particularly the epidermis (including the epidermal basal layer), with some penetration into the dermis. It is thought that the high concentration of dissolved cannabinoids on the outer surface of the skin causes a concentration gradient that enhances penetration of the cannabinoid into the skin, particularly the epidermis and the dermis.

In contrast to the prior art, the present invention provide a regime for use in the treatment or prevention of psoriasis, said regime comprising the administration of:
a) between 50 mg and 3000 mg of a topical composition comprising between 1% w/w and 15% w/w cannabinoid to the skin of a subject in need of such treatment or prevention.

Preferably the topical composition comprising between 1% w/w and 15% w/w cannabinoid is a liquid or gel composition.

Preferably, an amount of between 50 mg and 3000 mg, between 50 mg and 2000 mg, between 50 mg and 1000 mg, between 50 mg and 500 mg, between 50 mg and 400 mg, between 50 mg and 300 mg, between 50 mg and 200 mg, between 50 mg and 100 mg of the composition may be administered to the skin of the subject in each administration. For example, 50 mg, 100 mg, 200 mg, 300 mg, 400 mg, 500 mg, 600 mg, 700 mg, 800 mg, 900 mg, 1000 mg, 1500 mg, 2000 mg, 2500 mg or 3000 mg of the composition may be administered to the skin of the subject in each administration. Preferably an amount of about 100 mg is administered to the skin of the subject in each administration.

Preferably, an amount of between 50 mg and 3000 mg, between 50 mg and 2000 mg, between 50 mg and 1000 mg, between 50 mg and 500 mg, between 50 mg and 400 mg, between 50 mg and 300 mg, between 50 mg and 200 mg, between 50 mg and 100 mg of the composition may be administered to the face of the subject in each administration. For example, 50 mg, 100 mg, 200 mg, 300 mg, 400 mg, 500 mg, 600 mg, 700 mg, 800 mg, 900 mg, 1000 mg, 1500 mg, 2000 mg, 2500 mg or 3000 mg of the composition may be administered to the face of the subject in each administration. Preferably an amount of about 100 mg is administered to the face of the subject in each administration.

Preferably, an amount of between 50 mg and 3000 mg, between 50 mg and 2000 mg, between 50 mg and 1000 mg, between 50 mg and 500 mg, between 50 mg and 400 mg, between 50 mg and 300 mg, between 50 mg and 200 mg, between 50 mg and 100 mg of the composition may be administered to 565 cm² of skin of the subject in each administration. For example, 50 mg, 100 mg, 200 mg, 300 mg, 400 mg, 500 mg, 600 mg, 700 mg, 800 mg, 900 mg, 1000 mg, 1500 mg, 2000 mg, 2500 mg or 3000 mg of the composition may be administered to 565 cm² of skin of the subject in each administration. Preferably an amount of about 100 mg is administered to 565 cm² of the subject in each administration.

Preferably the composition comprises between 1% w/w and 15% w/w cannabinoid, between 1% w/w and 14% w/w, between 1% w/w and 13% w/w, between 1% w/w and 12% w/w, between 1% w/w and 11% w/w, between 1% w/w and 10% w/w, between 1% w/w and 9% w/w, between 1% w/w and 8% w/w, between 1% w/w and 7% w/w, between 1% w/w and 6% w/w, between 1% w/w and 5% w/w, between 2% w/w and 5% w/w, between 2% w/w and 4% w/w, between 3% w/w and 5% w/w, between 4% w/w and 5% w/w cannabinoid. For example, the composition may comprise 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, or 15% w/w cannabinoid

In certain embodiments, the concentration of cannabinoid in the topical composition of the invention may be selected from the group consisting of: at least 2% w/w, at least 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w, and at least 15% w/w.

In certain embodiments, the concentration of cannabinoid in the topical composition may be within a range with a lower limit selected from the group consisting of: 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, and 15% w/w; and an upper limit selected from the group consisting of: 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w and 15% w/w.

More preferably, the concentration of cannabinoid in the topical composition is between 2% w/w and 25% w/w cannabinoid, more preferably 5% w/w, 10% w/w or 20% w/w.

Preferably, the composition of the treatment regime delivers between 20 mg and 400 mg of cannabinoid per administration. For example, the composition of the treatment regime deliver may between 20 mg and 400 mg, 20 mg and 350 mg, 20 mg and 300 mg, 20 mg and 250 mg, 20 mg and 200 mg, 20 mg and 150 mg, 20 mg and 100 mg, 20 mg and 50 mg, 30 mg and 100 mg, 40 mg and 100 mg, 50 mg and 100 mg, 60 mg and 100 mg, 70 mg and 100 mg, 80 mg and 100 mg of cannabinoid per administration.

In certain embodiments, the composition of the treatment regime delivers an amount of cannabinoid per administration with a lower limit selected from the group consisting of: 20 mg, 30 mg, 40 mg, 50 mg, 60 mg, 70 mg, 80 mg, 90 mg, 100 mg, 110 mg, 120 mg, 130 mg, 140 mg, 150 mg, 200 mg, 250 mg, 300 mg and 350 mg; and an upper limit selected from the group consisting of: 30 mg, 40 mg, 50 mg, 60 mg, 70 mg, 80 mg, 90 mg, 100 mg, 110 mg, 120 mg, 130 mg, 140 mg, 150 mg, 200 mg, 250 mg, 300 mg, 350 mg and 400 mg.

More preferably, the amount of cannabinoid per administration is 27.5 mg, 37.5 mg or 75 mg.

In accordance with certain embodiments, the composition is applied to the affected area regularly until relief is obtained. In one preferred embodiment, the composition is administered to the skin of the patient in need of such treatment using a dosing regimen selected from the group consisting of: every hour, every 2 hours, every 3 hours, once daily, twice daily, three times daily, four times daily, five times daily, once weekly, twice weekly, once fortnightly and once monthly. However, other application schedules may be utilized in accordance with the present invention. Preferably, the composition of the treatment regime is administered to the skin between 1 and 5 times per day, more preferably once or twice per day.

Preferably the total daily dose applied to the skin by administration of the topical composition is between 20 mg and 2000 mg cannabinoid, preferably 20 mg and 2000 mg, 50 mg and 1500 mg, 100 mg and 1000 mg, 150 mg and 500 mg, 200 mg and 500 mg, 200 mg and 400 mg of cannabinoid.

In certain embodiments, the total daily dose of cannabinoid applied to the skin by administration of the topical composition has a lower limit selected from the group consisting of: 20 mg, 30 mg, 50 mg, 70 mg, 100 mg, 150 mg, 200 mg, 210 mg, 220 mg, 230 mg, 240 mg, 250 mg, 260 mg, 270 mg, 280 mg, 290 mg, 300 mg, 320 mg, 350 mg, 400 mg, 500 mg, 600 mg, 700 mg, 800 mg, 900 mg, 1000 mg, 1500 mg and 1900 mg; and an upper limit selected from the group consisting of: 30 mg, 50 mg, 70 mg, 100 mg, 150 mg, 200 mg, 210 mg, 220 mg, 230 mg, 240 mg, 250 mg, 260 mg, 270 mg, 280 mg, 290 mg, 300 mg, 320 mg, 350 mg, 400 mg, 500 mg, 600 mg, 700 mg, 800 mg, 900 mg, 1000 mg, 1500 mg and 2000 mg.

Most preferably, the total daily dose of cannabinoid applied to the skin by administration of the topical composition is 27.5 mg, 55 mg, 75 mg or 110 mg.

Thus in relation to the compositions of the present invention, preferably:
an amount of between 50 mg and 3000 mg of the composition is administered to the skin;
the administered composition contains between 1% and 15% cannabinoid;
the administered composition delivers between 20 mg and 400 mg cannabinoid;
the composition is administered between 1 and 5 times per day; and
the total daily dose applied to the skin is between 20 mg and 2000 mg cannabinoid.

More preferably:
an amount of between 100 mg and 120 mg of the composition is administered to the skin;
the administered composition contains between 2% w/w and 25% w/w cannabinoid;
the administered composition delivers between 20 mg and 150 mg cannabinoid;
the composition is administered one or two times per day; and
the total daily dose applied to the skin is between 20 mg and 200 mg cannabinoid.

Most preferably:
an amount of between 100 mg and 120 mg of the composition is administered to the skin;
the administered composition contains 5% w/w, 10% w/w or 20% w/w cannabinoid;
the administered composition delivers 27.5 mg, 37.5 mg or 75 mg cannabinoid;
the composition is administered one or two times per day; and
the total daily dose applied to the skin is 27.5 mg, 55 mg, 75 mg or 110 mg cannabinoid.

High concentrations of cannabinoids delivered to the skin are expected to be advantageous in terms of enhancing the relevant extent of delivery into the skin, particularly the epidermis (including the epidermal basal layer), with some penetration into the dermis. It is thought that the high concentration of cannabinoids on the outer surface of the skin causes a concentration gradient that enhances penetration of the cannabinoid into the skin, particularly the epidermis and the dermis.

In order to achieve local distribution for the treatment of psoriasis, it is advantageous for the majority of the cannabinoid, such as cannabidiol (CBD), to penetrate into the epidermis and preferably remain there, and for some cannabinoid to further penetrate to the dermis and the hypodermal layer to be absorbed systemically. In such a case, the cannabidiol would concentrate mainly in the epidermis, thus maximizing its local effect. Not only does the localized effect increase the potential therapeutic benefit, it potentially lessens the frequency and severity of any potential side-effects associated with systemic cannabinoid administration, because the amount of active compound circulating in the patient is reduced.

### Treatment and Prevention of Psoriasis

In certain embodiments the topical application of cannabinoid, such as cannabidiol, by way of the compositions of the present invention is expected to reduce the incidence and/or severity of psoriasis. Therapeutic effects of the present invention include, but are not limited to, reduction in redness, itch, pain or irritation, a reduction in blisters or pustules, a reduction in infection, a reduction of swelling, cracking, weeping, crusting, and scaling and/or a general decrease in inflammation.

In certain embodiments, the topical application of cannabinoid, such as cannabidiol, by way of the compositions of the present invention is expected to improve the symptoms of psoriasis.

The term “improve” is used to convey that the present invention changes either the appearance, form, characteristics and/or the physical attributes of the tissue to which it is being provided, applied or administered. The change in form may be demonstrated by any of the following alone or in combination: enhanced appearance of the skin; decreased inflammation of the skin, prevention of inflammation or blisters, decreased spread of blisters, decreased ulceration of the skin, decreased redness, reduction of scarring, reduction in lesions, healing of blisters, reduced skin thickening, closure of wounds and lesions, a reduction in symptoms including, but not limited to, pain, inflammation, itching, milia or other symptoms associated with inflammatory conditions or the like.

A primary advantage of the present invention is expected to be the improvement in the condition of the skin without the typical side effects of conventional therapies. The potential for the present invention is widespread, and the topical application of cannabinoids shows promise as an exciting new method of psoriasis treatment.

It is expected that treatment of psoriasis in accordance with embodiments of the present invention results in improved healing of the skin. For example, when used in the treatment of psoriasis, swollen, cracked or scaled skin is which is treated is expected to heal more quickly and/or completely, compared to when left untreated.

When administered in accordance with the present invention, treatment is expected to result in one or more therapeutic effects. Therapeutic effects in the affected area include, but are not limited to, reduction in redness, itch, pain or irritation, the number and severity of the psoriatic lesions, a reduction in infection, a reduction of swelling, cracking, weeping, crusting, and scaling and/or a general decrease in inflammation. One or more of these therapeutic effects are expected to be observed when treatment in accordance with the present invention is made to any of the suitable conditions.

The present invention therefore provides a method for treating or preventing psoriasis, said method comprising the administration of:
a) between 50 mg and 3000 mg of a topical composition comprising between 1% w/w and 15% w/w cannabinoid to the skin of a subject in need of such treatment or prevention.

Preferably the topical composition comprising between 1% w/w and 15% w/w cannabinoid is a liquid or gel composition. Preferably the composition is non-aqueous.

The present invention further provides for the use of between 50 mg and 3000 mg of a topical composition comprising between 1% w/w and 15% w/w cannabinoid for the treatment or prevention of psoriasis in a subject in need of such treatment or prevention.

The present invention further provides for the use of between 1% w/w and 15% w/w cannabinoid for the manufacture of a topical composition for the treatment or prevention of psoriasis, wherein between 50 mg and 3000 mg of the topical composition is administered to the skin of a subject in need of such treatment or prevention.

In one aspect, the present invention is directed to methods of treating or preventing psoriasis using topical cannabinoids, including cannabidiol. In accordance with certain embodiments, a topical composition of the invention containing cannabinoids such as cannabidiol, is preferably applied topically to an area which is affected by the psoriasis. Preferably, the application of cannabinoid in accordance with certain embodiments results in reduction in redness, itch, pain or irritation, a reduction in blisters or pustules, a reduction in infection, less breakdown and loss of collagen and elastin in the skin, a reduction of swelling, cracking, weeping, crusting, and scaling and/or a general decrease in inflammation.

Thus in relation to the methods of the present invention, preferably:
an amount of between 50 mg and 3000 mg of the composition is administered to the skin;
the administered composition contains between 1% and 15% cannabinoid;
the administered composition delivers between 20 mg and 400 mg cannabinoid;
the composition is administered between 1 and 5 times per day; and
the total daily dose applied to the skin is between 20 mg and 2000 mg cannabinoid.

More preferably:
an amount of about 100 mg of the composition is administered to the skin;
the administered composition contains between 2% w/w and 25% w/w cannabinoid;
the administered composition delivers between 20 mg and 150 mg cannabinoid;
the composition is administered one or two times per day; and
the total daily dose applied to the skin is between 20 mg and 200 mg cannabinoid.

Most preferably:
an amount of between 100 mg and 120 mg of the composition is administered to the skin;
the administered composition contains 5% w/w, 10% w/w or 20% w/w cannabinoid;
the administered composition delivers 27.5 mg, 37.5 mg or 75 mg cannabinoid;
the composition is administered one or two times per day; and
the total daily dose applied to the skin is 27.5 mg, 55 mg, 75 mg or 110 mg cannabinoid.

### Pharmaceutical Composition

The present invention provides a composition comprising between 1% w/w and 15% w/w cannabinoid for use in the treatment or prevention of psoriasis, wherein between 50 mg and 3000 mg of the topical composition is administered to the skin of a subject in need of such treatment or prevention. Preferably the composition is administered to the skin between 1 and 5 times per day and preferably the total daily dose applied to the skin by administration of the topical composition is between 20 mg and 2000 mg cannabinoid.

Preferably there is a therapeutically effective amount of cannabinoid in each topical dose of the composition of the present invention. Therapeutically effective amount means the amount necessary to bring about a therapeutic effect.

Certain embodiments of the present invention comprise any topically acceptable carrier vehicle. Preferred topically acceptable vehicles include but are not limited to gels, ointments, and liquids. Administration of the preferred embodiment is performed in accordance with that mode which is most amenable to the topically acceptable form chosen. For example, gels, lotions, creams and ointments are preferably administered by spreading. The topical composition may or may not contain water, i.e. it may be an aqueous or a non-aqueous composition.

The dilution of the cannabinoid in the topical composition can be an important consideration. The cannabinoid concentration in the composition should be high enough that the patient does not need to wait an excessively long time for the composition to dry. On the other hand, the cannabinoid concentration should be dilute enough that a patient can achieve effective coverage of the affected area. Additionally, the composition could include a component which polymerizes in response to exposure to air or ultraviolet radiation.

The amount of composition to be applied will vary. When the cannabinoid, such as cannabidiol, is administered by spraying a solution of the drug, the total volume in a single dose may be as low as 0.1 ml. When the cannabinoid, such as cannabidiol, is administered in a gel or cream, the total volume may be as high as 3 ml. Conversely, if the psoriasis comprises scattered lesions, the volume applied to each lesion may be smaller. The carrier selected, and its manner of application, are preferably chosen in consideration of the needs of the patient and the preferences of the administering physician.

In one preferred embodiment, the composition comprises a gel which is preferably administered by spreading the gel onto the affected area. In other preferred embodiments, the composition comprises a liquid, which can be administered by spraying or otherwise applying the liquid onto the affected area.

In certain embodiments, the composition of the invention may be provided in a form selected from the group comprising, but not limited to a liquid, cream or gel. The composition may be a leave-on preparation or a wash-off preparation. In one preferred form, the composition is a cream or gel. In another preferred form, the composition is a spray. The composition may or may not contain water. Preferably, the composition does not contain water, i.e. it is non-aqueous.

The cannabinoid could be incorporated into a composition with an additional active moiety that is capable of improving the appearance and/or hydration of the skin.

In addition, the composition of the present invention can be used in conjunction with other topically applied analgesic and/or systemically available agents for the treatment of psoriasis.

Examples of such analgesic agents include, but are not limited to: morphine, cyclazocine, piperidine, piperazine, pyrrolidine, morphiceptin, meperidine, trifluadom, benzeneacetamine, diacylacetamide, benzomorphan, alkaloids, peptides, phenantrene and pharmaceutically acceptable salts, prodrugs or derivatives thereof. Specific examples of compounds contemplated by as suitable in the present invention include, but are not limited to morphine, heroin, hydromorphone, oxymorphone, levophanol, methadone, meperidine, fentanyl, codeine, hydrocodone, oxycodone, propoxyphene, buprenorphine, butorphanol, pentazocine and nalbuphine. As used in the context of opioid agents herein, “pharmaceutically acceptable salts, prodrugs and derivatives” refers to derivatives of the opioid analgesic compounds that are modified by, e.g., making acid or base salts thereof, or by modifying functional groups present on the compounds in such a way that the modifications are cleaved, either in routine manipulation or in vivo, to produce the analgesically active parent compound. Examples include but are not limited to mineral or organic salts of acidic residues such as amines, alkali or organic salts of acidic residues such as carboxylic acids, acetate, formate, sulfate, tartrate and benzoate derivatives, etc. Suitable opioid analgesic agents, including those specifically mentioned above, are also described in Goodman and Gilman, ibid, chapter 28, pp. 521-555.

In addition, other active agents may be included in the composition of the present invention, e.g., topically-effective anaesthetics such as xylocaine, cocaine, lidocaine, benzocaine, etc., which may provide a more immediate, if less effective in the long run, level of pain relief until the analgesic agent becomes fully effective.

Still other agents can also be administered, preferably topically, to potentiate the effects of the topically-administered cannabidiol. For example, dextromethorphan, a non-addictive opioid compound, can be co-administered, preferably topically, although parenteral administration is also effective, to enhance the effectiveness of the topically administered agent. Without wishing to be bound by theory, it is believed that dextromethorphan has previously unappreciated analgesic properties in peripheral nerves. Suitable concentrations of dextromethorphan are routinely ascertainable by the skilled worker, and include the normal therapeutic amounts administered parenterally for conventional purposes, e.g., as a cough suppressant, or less, and routinely determinable amounts for topical administration; for example, 1 g of dextromethorphan can be added to a composition disclosed herein to provide additional treatment for psoriasis.

In one embodiment, the pharmaceutical composition of the present invention further comprises one or more of the following agents for the treatment of psoriasis: salicylic acid; resorcinol; sulfacetamide; urea; imidazoles such as ketoconazole and elubiol; essential oils; alpha-bisabolol; dipotassium glycyrrhizinate; camphor; beta.-glucan; allantoin; feverfew; flavonoids such as soy isoflavones; saw palmetto; chelating agents such as EDTA; lipase inhibitors such as silver and copper ions; hydrolyzed vegetable proteins; inorganic ions of chloride, iodide, fluoride, and their nonionic derivatives chlorine, iodine, fluorine; synthetic phospholipids and natural phospholipids; steroidal anti-inflammatory agents such as hydrocortisone, hydroxyltriamcinolone alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, fluticasone monopropionate, fluticasone furoate, mometasone furoate, budesonide, ciclesonide and salts are prodrugs thereof; nonsteroidal anti-Inflammatory drugs (NSAIDs) such as COX inhibitors, LOX inhibitors, p38 kinase inhibitors including ibuprofen, naproxen, salicylic acid, ketoprofen, hetprofen and diclofenac; analgesic active agents for treating pain and itch such as methyl salicylate, menthol, trolamine salicylate, capsaicin, lidocaine, benzocaine, pramoxine hydrochloride, and hydrocortisone; antibiotic agents such as mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, tetracycline, clindamycin, erythromycin; immunosuppressant agents such as cyclosporine and cytokine synthesis inhibitors, tetracycline, minocycline, and doxycycline, or any combination thereof.

In preferred forms of the invention, the formulation is not a solid formulation, such as a patch or adhesive bandage. In preferred forms of the invention, the composition is a liquid formulation.

It is preferable that the composition concentrates the cannabinoid on the skin. To achieve this, one preferred method is to provide the cannabinoid in a composition comprising a mixture of a volatile solvent and a residual (less volatile) solvent.

### Volatile Solvents

By using a volatile solvent, one can achieve much higher, non-crystalline (i.e., in solution), concentrations of cannabinoids. The cannabinoids can be dissolved in much higher concentrations of the volatile solvent, and then once applied to the skin and the volatile solvent has evaporated, the cannabinoids remain on the skin in high concentrations. The volatile solvent may, for example, be a C₂₋₆ low molecular weight alcohol such as methanol, isopropanol, propanol, 2-butanol, n-butanol and ethanol. Alternatively, the volatile solvent may be a siloxane. Other suitable volatile solvents will be clear to the skilled reader.

In a preferred form of the invention, the composition comprises a combination of a C₂₋₆ low molecular weight alcohol and a siloxane.

Advantageously, in some embodiments, the volatile solvent is a liquid at ambient temperatures. Preferably the volatile solvent is liquid at about 30° C., or less, or at about 25° C. Preferably the level of volatility of the volatile solvent is about the same as that of isopropyl alcohol. Preferably, the boiling point of the volatile solvent is between about 70° C. and 110° C. at atmospheric pressure. Preferably, the boiling point of the volatile solvent is between about 80° C. and 105° C. at atmospheric pressure. Preferably, the boiling point of the volatile solvent is between about 85° C. and 105° C. at atmospheric pressure.

Advantageously, in some embodiments, the volatile solvent is selected from the group consisting of: C₂₋₆ alcohols, and combinations thereof. Advantageously, in some embodiments, the volatile solvent is selected from the group consisting of: C₂₋₄ alcohols, and combinations thereof. In specific embodiments, the volatile solvent is selected from the group consisting of: ethyl alcohol (or ethanol), n-propanol, isopropyl alcohol, butanol, transcutol, and combinations thereof. Other volatile solvents will be clear to the skilled reader.

Alternatively, the volatile solvent comprises a siloxane. Preferably, the volatile solvent comprises a non-polymeric siloxane.

In a preferred form of the invention, the siloxane contains from one to eight silicon atoms per molecule. In a preferred form of the invention, the siloxane contains from two to five silicon atoms per molecule. In one embodiment, the siloxane contains two or three silicon atoms.

The siloxanes may have between one and eight methyl groups. In one embodiment, the siloxane is selected from the group consisting of: hexamethyldisiloxane, octamethyltrisiloxane and combinations thereof. These are the most volatile siloxanes, and are thus the most advantageous. Preferably the level of volatility of the siloxane is about the same as that of isopropyl alcohol.

In another embodiment, the siloxane contains 4 or 5 silicon atoms, and is, for example, decamethyltetrasiloxane or dodecamethylpentasiloxane. In another embodiment, the siloxane is a cyclical 4 or 5 silicon atom compound such octamethylcyclotetrasiloxane (CAS#556-67-2) or decamethylcyclopentasiloxane (CAS#541-02-6).

In one form of the invention, the volatile solvent is hexylmethyldisiloxane which is combined with less volatile polymethylsiloxane.

In a preferred form of the invention, the composition comprises a combination of a C₂₋₆ low molecular weight alcohol and a non-polymeric siloxane.

In a preferred form of the invention, the cannabinoid is dissolved in the volatile solvent.

In specific embodiments, the relative amount of volatile solvent is selected from the following group: at least 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, 95% w/w or 97% w/w. In specific embodiments, the maximum concentration of the volatile solvent is 50% w/w, 60% w/w, 70% w/w, 80% w/w, 90% w/w, 95% w/w or 97% w/w. The relative amount of volatile solvent may be between 1% w/w and 97% w/w, 10% w/w and 97%, 10% w/w and 90% w/w, 50% w/w and 97% w/w, 50% w/w and 95% w/w.

Preferably, the volatile solvent is provided as:
85-95% w/w non-polymeric siloxane;
85-95% w/w non-polymeric siloxane and 1-10% wt/wt C₂-C₆ alcohol;
70-80% w/w wt/wt C₂-C₆ alcohol.

The C₂-C₆ alcohol may be transcutol, another C₂-C₆ alcohol (such as isopropyl alcohol) or a mixture of transcutol and another C₂-C₆ alcohol (such as isopropyl alcohol).

### Residual Solvents

The cannabinoids are preferably kept in a non-crystalline form on the skin after evaporation of the volatile solvent by the addition of a less volatile solvent. This less volatile solvent is called the residual solvent, as it may remain on the skin after evaporation of the volatile solvent to keep the cannabinoid in a non-crystalline state after evaporation of the volatile solvent. Preferably the residual solvent has a low volatility such that less than 5% would evaporate at skin temperature over 24 hours. Preferably, the residual solvent has a chain structure that has a hydrophobic end and a hydrophilic end. Preferably the residual solvent is a liquid at or below 32° C. Preferably the residual solvent dissolves the volatile solvent. Preferably the residual solvent maintains the cannabinoid in non-crystalline form, i.e. in solution, at concentrations of 20% up to 70% w/w cannabinoid.

The purpose of the residual solvent is to act as a solvent for the cannabinoid once the volatile solvent has evaporated. The residual solvent may be a compound from the list comprising: fatty acids, fatty acid alcohols, fatty alcohols, glycols or alkanes, or ethers of any of these. It is preferably a C₁₂₋₂₂ compound. The residual solvent may comprise a mixture of, for example, alkyl polypropylene glycol/polyethylene glycol ether and/or a fatty acid alcohol and/or a fatty alcohol. In specific embodiments the residual solvent is a C₁₂₋₂₂ fatty alcohol. In specific embodiments, the residual solvent is a C₁₆₋₂₂ fatty alcohol. In specific embodiments, the residual solvent is selected from the group consisting of: oleyl alcohol, isostearyl alcohol, isohexadecane, octyldodecyl alcohol, 2-hexyl decyl alcohol. Most preferably the residual solvent is isohexadecane.

In specific embodiments, the relative amount of residual solvent may be selected from the following group: at least 1% w/w, at least 2% w/w, at least 3% w/w, at least 4% w/w, at least 5% w/w, at least 6% w/w, at least 7% w/w, at least 8% w/w, at least 9% w/w, at least 10% w/w, at least 20% w/w, at least 30% w/w, at least 40% w/w, at least 50% w/w. In specific embodiments, the maximum concentration of the residual solvent is 50% w/w. In specific embodiments, the maximum concentration of the residual solvent is 80% w/w. The relative amount of residual solvent may be selected from the following group: between 1% and 80% w/w, between 1% and 50% w/w, between 1% and 40% w/w, between 1% and 30% w/w, between 1% and 20% w/w, between 1% and 10% w/w, between 2% and 80% w/w, between 2% and 50% w/w, between 1% and 20% w/w, between 2% and 10% w/w. Preferably the amount of residual solvent is between 1-15% w/w.

Preferably the amount of residual solvent is sufficient to keep the cannabinoid in a non-crystalline form, i.e. in solution, on the skin after partial or complete evaporation of the more volatile solvent or solvents.

Where the composition comprises a residual solvent and a volatile solvent, the composition comprises a solution of the cannabinoid in the mixture of the volatile solvent and the residual solvent. The composition may consist of a solution of the cannabinoid in the mixture of the volatile solvent and the residual solvent, or comprise a solution of the cannabinoid in the mixture of the volatile solvent and the residual solvent in combination with solid cannabinoid, such as a suspension of solid cannabinoid in a saturated solution of the cannabinoid in the mixture of volatile solvent and residual solvent. In preferred forms of the invention, the composition does not comprise solid cannabinoid.

The total amount of the volatile solvent, and the residual solvent if present, required is sufficient to keep the cannabinoid non-crystalline, i.e. in solution, at room temperature for between about 2-8 hours once the composition is applied to the skin.

The preferred ratio of cannabinoid to siloxane to residual solvent is selected from the range consisting of (w/w %):
between 0.5-20% cannabinoid, between 1-99% siloxane and between 0.1-98.5% residual solvent;
between 5-20% cannabinoid, between 4-70% siloxane and between 1%-70% residual solvent; or
between 1-10% cannabinoid, between 20-98% siloxane and between 1-15% residual solvent.

The preferred ratio of cannabinoid to hexamethyldisiloxane to residual solvent is selected from the range consisting of (w/w %):
between 0.5-20% cannabinoid, between 1-99% hexamethyldisiloxane and between 0.1-98.5% residual solvent;
between 5-20% cannabinoid, between 4-70% hexamethyldisiloxane and between 1%-70% residual solvent; or
between 1-10% cannabinoid, between 20-98% hexamethyldisiloxane and between 1-15% residual solvent.

The preferred ratio of cannabinoid to 02-6 low molecular weight alcohol to residual solvent is selected from the range consisting of (w/w %):
between 0.5-20% cannabinoid, between 1-99% C₂₋₆ low molecular weight alcohol and between 0.1-98.5% residual solvent;
between 5-20% cannabinoid, between 4-70% C₂₋₆ low molecular weight alcohol and between 1%-70% residual solvent; or
between 1-10% cannabinoid, between 20-98% C₂₋₆ low molecular weight alcohol and between 1-15% residual solvent.

The preferred ratio of cannabinoid to transcutol/isopropyl alcohol to residual solvent is selected from the range consisting of (w/w %):
between 0.5-20% cannabinoid, between 1-99% transcutol/isopropyl alcohol and between 0.1-98.5% residual solvent;
between 5-20% cannabinoid, between 4-70% transcutol/isopropyl alcohol and between 1%-70% residual solvent; or
between 1-10% cannabinoid, between 20-98% transcutol/isopropyl alcohol and between 1-15% residual solvent.

As noted above, in highly preferred forms of the invention, the composition comprises 5% w/w, 10% w/w or 20% w/w cannabidiol.

Where the composition contains 5% w/w, 10% w/w or 20% w/w cannabidiol, the composition preferably comprises 60-95% w/w volatile solvent in the form of: a non-polymeric siloxane and/or a C₂₋₆ low molecular weight alcohol.

In a preferred form of the invention, the C₂₋₆ low molecular weight alcohol is transcutol, another C₂-C₆ alcohol (such as isopropyl alcohol) or a mixture of transcutol and another C₂-C₆ alcohol (such as isopropyl alcohol).

In a preferred form of the invention, the non-polymeric siloxane comprises two to three silicon atoms per molecule. In a preferred form of the invention, the non-polymeric siloxane is hexamethyldisiloxane. In a preferred form of the invention, the viscosity of the siloxane, preferably hexamethyldisiloxane, is between 0.5 and 0.7 cSt.

Where the composition contains 5% w/w, 10% w/w or 20% w/w cannabidiol and 85-95% w/w volatile solvent in the form of a non-polymeric siloxane, the composition optionally further comprises a volatile solvent in the form of a 02-6 low molecular weight alcohol at a concentration of 1-10% w/w. In preferred forms of the invention, the concentration is 15% w/w. In preferred forms of the invention, the concentration is 2-4% w/w. In a preferred form of the invention, the 02-6 low molecular weight alcohol is an alcohol containing between two and four carbon atoms per molecule. In preferred forms of the invention, the 02-6 low molecular weight alcohol is isopropyl alcohol.

Where the composition contains 5% w/w, 10% w/w or 20% w/w cannabidiol, the composition optionally further comprises a volatile solvent in the form of a 02-6 low molecular weight alcohol at a concentration of 60-70% w/w. In a preferred form of the invention, the C₂₋₆ low molecular weight alcohol is an alcohol containing between two and four carbon atoms per molecule. In preferred forms of the invention, the 02-6 low molecular weight alcohol is transcutol and/or isopropyl alcohol.

Where the composition contains 5% w/w, 10% w/w or 20% w/w cannabidiol, 60-95% w/w volatile solvent (in the form of a non-polymeric siloxane and/or a 02-6 low molecular weight alcohol), the composition optionally further comprises 1-10% w/w residual solvent in the form of fatty acids, fatty acid alcohols, fatty alcohols, glycols, alkanes, ethers of any of these, and combinations thereof. In a preferred form of the invention, the residual solvent is isohexadecane.

### Viscosity Modifier

The present invention may include a viscosity modifier. The viscosity modifier has little effect on the delivery of the active cannabinoid from the composition, but may contribute significantly to patient compliance by improving the tactile qualities of the composition.

In one form of the invention, the viscosity modifier is a silicone fluid. In one form of the invention, the viscosity modifier is a polysiloxane. Where the viscosity modifier is a polysiloxane, the viscosity modifier is preferably a polydimethylsiloxane. Preferably, where the viscosity modifier is a polysiloxane, including a polydimethylsiloxane, the viscosity modifier has a viscosity of between 10,000 and 15,000 cSt, preferably still 11,500 and 13,500 cSt. In a highly preferred form of the invention, the viscosity modifier has a viscosity of approximately 12,500 cSt.

Where the polysiloxane viscosity modifier has a viscosity of between 10,000 and 15,000 cSt, the concentration of the polysiloxane viscosity modifier is preferably between 0.2 and 2% w/w. Preferably still, the concentration of the polysiloxane viscosity modifier is between 0.5 and 1.5% w/w. Preferably still, the concentration of the polysiloxane viscosity modifier is between 0.8 and 1.2% w/w.

The polysiloxane viscosity modifier may be provided in the form of a dimethiconol gum. The dimethiconol gum may be used alone, or in conjunction with another polysiloxane viscosity modifier, such as polydimethylsiloxane. In preferred forms of the invention, the dimethiconol gum is used in conjunction with the polydimethylsiloxane viscosity modifier. Preferably, the concentration of the dimethiconol gum viscosity modifier in the composition is between 3 and 7% w/w. Preferably, the concentration of the dimethiconol gum viscosity modifier in the composition is between 4 and 6% w/w. Preferably, the concentration of the dimethiconol gum viscosity modifier in the composition is between 4.5 and 5.5% w/w.

Such administration is expected to result in enhanced delivery of a cannabinoid, such as cannabidiol, to the epidermis and dermis of the skin, which is expected to be effective in significantly reducing, and therefore treating, acne in patients in need of such treatment.

In one preferred embodiment, the composition is non-aqueous. In another preferred embodiment, the composition does not comprise a preservative.

### General

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Those skilled in the art will appreciate that the invention described herein is susceptible to variations and modifications other than those specifically described. The invention includes all such variation and modifications. The invention also includes all of the steps, features, compositions and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The invention described herein may include one or more range of values (e.g. concentration). A range of values will be understood to include all values within the range, including the values defining the range, and values adjacent to the range which lead to the same or substantially the same outcome as the values immediately adjacent to that value which defines the boundary to the range.

### EXAMPLES

Further features of the present invention are more fully described in the following description of several non-limiting embodiments thereof. The following Examples are to be construed as merely illustrative and not limitative of the remainder of the disclosure in any way whatsoever. This description is included solely for the purposes of exemplifying the present invention. It should not be understood as a restriction on the broad summary, disclosure or description of the invention as set out above.

### Example 1

### Techniques for Ascertaining Permeability of Compositions Containing Cannabidiol

Dermatomed skin from a single donor was mounted in a Franz-type diffusion cell (0.55 cm² receptor fluid exposure surface area) and dosed with 5 ul of CBD formulated in an mixture of a volatile solvent (hexylmethyldisiloxane/polymethylsiloxane—93% w/w), and residual solvent (arlamol E—2% w/w) at a concentration of 5.0% (w/w; 35.5 mg/ml). Following dosing, receptor phase samples were collected at 4, 10, 24 and 48 hours; after which the study was terminated.

The residual formulation was removed by tape stripping and the epidermis and dermis separated by blunt dissection. The levels of CBD in the epidermis, dermis, and receptor fluid samples were then analyzed using a bioanalytical method with LC-MS/MS detection.

The data showed that skin permeation (i.e., permeation through to the receptor phase of the test system) was negligible, with less than 0.081% (278 ng/cm²) in the receptor phase over the 48-hour exposure period.

The various layers of the skin showed different amounts of absorbed dose over the 48-hour period: epidermal deposition of CBD was 13.17% of the applied dose, while dermal deposition of CBD was 4.54% of the applied dose. The dermis concentration was 8,408 ng/cm² or 1,933 ng/g of tissue (˜1,933 ng/mL) following application of CBD mixture.

These results suggest that the level of systemic exposure for CBD is likely to be very low following topical administration in vivo.

### Example 2

Vehicle and Comparator-Controlled, Evaluator-Blinded Trial to Evaluate the Safety and Anti-Psoriatic Efficacy of Topical Formulations of BTX 1308 in Subjects with Psoriasis Vulgaris in a Psoriasis Plaque Test

This will be single-centre, randomised, evaluator-blinded, vehicle- and active-comparator controlled, dose-finding study. The objective of this proof-of-concept study is to assess preliminary safety, tolerability, and activity of various formulations and concentrations of BTX 1308 in subjects with mild to moderate, stable, plaque-type psoriasis. The subjects will have target lesion(s) on the trunk or extremities (excluding palms/soles); psoriatic lesions on the knees or elbows (joints) are not to be used as target lesions. Subjects will have identified treatment fields with a comparable EPB thickness of the psoriatic infiltrate of at least 200 μm. This will be measured on Day 1.

Approximately 15 subjects will be enrolled. The subjects can be of either gender, with stable mild to moderate psoriasis vulgaris in a stable phase in up to 3 plaque(s) with an area sufficient for 6 test fields.

Subjects should not:
have acute guttate psoriasis, erythrodermatic psoriasis or pustular psoriasis;
have used systemic treatment with anti-psoriatics e.g., corticosteroids, cytostatics, or retinoids in the three months before the Baseline Visit;
have used biologicals (e.g., ustekinumab, secukinumab, ixekizumab, guselkuma, adalimumab, infliximab and etanercept) within six months before the Baseline Visit;
have used UV-therapy within four weeks before first treatment.

Each subject will have six (6) treatments applied. Topical occlusive applications of approximately 200 μL per test field (1.1 cm2) will be done once daily during a 19-day study period, except on Days 7, 14, and 19, for a total of 16 treatments.

Study drug will be applied topically to plaque psoriasis on the trunk or extremities located on up to 3 comparable psoriatic plaque(s) adequate to treat 6 separate fields (min 1.5 cm apart). Approximately 200 μL of each study drug will be applied occlusively to the 6 test fields.

Multiple formulations of BTX 1308 with the active ingredient cannabidiol (CBD) will be tested:
BTX 1308-1 5% CBD
BTX 1308-2 10% CBD
BTX 1308-3 20% CBD
BTX 1308-5 20% CBD
BTX 1308 Vehicle with no CBD
Betamethasone valerate (Betnovate® 0.1% Ointment)

The excipients include hexamethyldisiloxane, dimethicone, polypropylene glycol-15 (PPG-15) stearyl ether, isopropyl alcohol (IPA) and Transcutol P (diethylene glycol monoethyl ether) which have been used extensively in other topical products. The active BTX 1308 study products are a clear to light yellow solution with a 5%, 10% or 20% (w/w) concentration of CBD. The compositions of the BTX 1308 formulations and vehicle-control are presented in Table 2.

TABLE 2
Composition of the BTX 1308 Formulations

| | BTX 1308 Formulation ID | | | | |
|---|---|---|---|---|---|
| | BTX | BTX | BTX | BTX | Vehicle- |
| Ingredients | 1308-1* | 1308-2 | 1308-3 | 1308-5 | control |
| Hexamethyldisiloxane | 91.8 | | | | 96.8 |
| Dimethicone | 1.1 | | | | 1.1 |
| Isopropyl Alcohol | — | 13.4 | 3.4 | 37.0 | |
| Transcutol P | — | 82.5 | 62.5 | — | |
| Polypropylene Glycol-15 | 2.1 | 14.1 | 14.1 | 43.0 | 2.1 |
| (PPG-15) Stearyl Ether | | | | | |
| Cannabidiol (CBD) | 5.0 | 10.0 | 20.0 | 20.0 | — |
| Total | 100.0 | 100.0 | 100.0 | 100.0 | 100.0 |

Commercially available Betnovate (betamethasone valerate) Ointment 0.1% will be purchased for the study. Daily exposure will be 200 μL of the Betnovate Ointment 0.1%, or 0.2 mg of betamethasone valerate. The table below describes the characteristics of the betamethasone valerate.

TABLE 3
Betamethasone Valerate

| Generic name/brand | Active comparator: |
|---|---|
| name/INN | Betnovate ointment, 0.1% |
| Formulation | Ointment |
| Active ingredient | Betamethasone valerate |
| Amount per unit | 0.1% |
| Additional ingredients | Viscous paraffin |
| Packaging | 25 g tube |
| Storage | ≤25° C. |
| Manufacturer | GlaxoSmithKline GmbH & Co. |
| | KG, Munich, Germany |

### Primary Efficacy Endpoint

The primary endpoint will be the change from Day 1 to Day 19 in psoriatic infiltrate thickness.

### Secondary Endpoints

Percent change from Baseline to Day 8, 12 and 19 in psoriatic infiltrate thickness
Change from Baseline to Day 8 and 12 in psoriatic infiltrate thickness
Change and percent change from Baseline to Day 8, 12 and 19 in erythema (chromametry)
Change from Baseline to Day 8, 12 and 19 of the AUC (area under the curve) of psoriatic infiltrate thickness
Evaluation of the anti-psoriatic efficacy by clinical assessment using a 5-point scale at Day 8, 12 and 19
Clinical assessment of erythema (abnormal redness of the skin; absolute score scale) using a 5-point scale at Day 8, 12 and 19.

### Other Exploratory Assessments

Photographic documentation of the test fields on Day 1 (pre-treatment) and Day 19.
A total of 7 biopsies of 4.5 mm diameter will be obtained from a subgroup of 5 subjects from each of 6 test fields on Day 19. One additional biopsy from untreated/otherwise normal skin will be obtained to serve as a control. The skin samples will be used for histology, immunohistochemistry, and gene expression analyses.

### Protocol

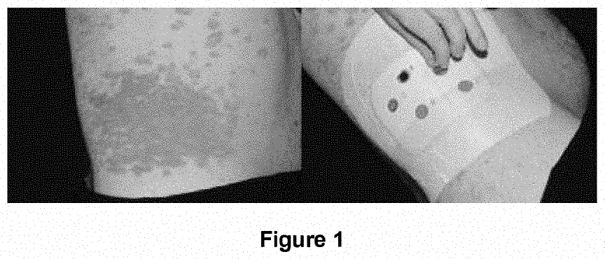

In this test system multiple formulations are applied topically to small (1.1 cm²) test fields that have been punched out of an occlusive bandage located over a psoriatic plaque (See FIG. 1). In the original test design described by Dumas and Scholtz in 1972 the action of corticosteroids on plaque-type psoriasis was evaluated based on graded visual assessments of the condition following treatment of up to 2 weeks. Since then, clinical assessments have be supplemented with more sensitive biophysical measurement methods such as chromametry, visiometry or high-frequency 22-MHz skin sonography to provide measurements of the inflammatory alterations accompanying psoriasis. The observation that the nonechogenic or echo poor band (EPB) as measured by 22-MHz sonography reflects the thickness of the psoriatic inflammatory infiltrate and this thickness correlates with the clinical severity of a psoriatic plaque, has opened up the use of EPB as a surrogate measurement for efficacy. The clinical relevance of the PPT and the measurement of EPB has been confirmed in a number of Phase 2 and Phase 3 clinical studies conducted with corticosteroids and vitamin D analogues.

Descaling of psoriasis plaques with salicylic acid (in petrolatum) will be done for a maximum of 5 days terminating Day −2. Serology will be done for subjects agreeing to biopsies on Day 19. Subjects will be monitored for AEs and changes in concomitant medications from time of consent through Day 19.

Once the subject has completed descaling with salicylic acid and continues to be deemed eligible, they may be enrolled and begin Day 1 (Baseline) assessments (within 28 days after the Screening Visit).

The test fields will be identified and marked with a skin marker. One of multiple (up to 3) hydrocolloid dressings will be prepared based on the locations of the test fields. The hydrocolloid dressing will be fixed on the skin prior to study drug application with adhesive patches (BSN, Hamburg, Germany, or comparable).

Approximately 200 μL of each study drug will be applied to each test field (6 in total) using special test chambers (Duhring chambers, 12 mm inside 0, 14 mm outside 0). Six holes corresponding to the identified fields will be punched in one or multiple hydrocolloid dressing(s) (Varihesive® E). Cotton webril pads will be inserted into each of 6 special aluminum chambers (Duhring chambers). Approximately 200 μL of each study drug will be applied to the cotton webril pad and the Duhring chambers will be immediately seated in holes punched in a hydrocolloid dressing. The chambers will then be fixed in place with Leukosilk®. Each treatment field is 1.1 cm2 in area.

The chambers will be removed before each new application. In previous studies the hydrocolloid dressing has been shown to be well tolerated. The distance between the chambers must be at least 1.5 cm. This distance is sufficient to exclude interactions with neighbouring fields. The fields will be treated occlusively for a treatment period of 19 days. On 16 of the trial days treatments will be performed (Days 1-6, 8-13, 15-18). Before each new application remaining preparation residues will be removed by gently cleansing each test field with a separate soft tissue. The hydrocolloid dressing will be renewed before sonographic measurement on Days 8 and 12, or at other treatment days, if necessary. In case of a missed day of treatment/evaluation, the subject is advised to keep the Duhring chamber patches on the plaques until he/she can return to the clinical centre. Dressings and plasters must not be removed by the subject. If they fall off they should not be put back on. Instead they should be returned to the trial centre at the next visit and the approximate time the dressing/plaster fell off should be reported. The subject should return to the clinical centre at the next scheduled dosing occasion. Trial procedures performed will be such that any procedures that were to be performed on the missed day are performed at the next scheduled visit if not already planned

The dose for all subjects will be 200 μL of each study drug to the 1.1 cm² treatment field per day. No escalation of dose will occur.

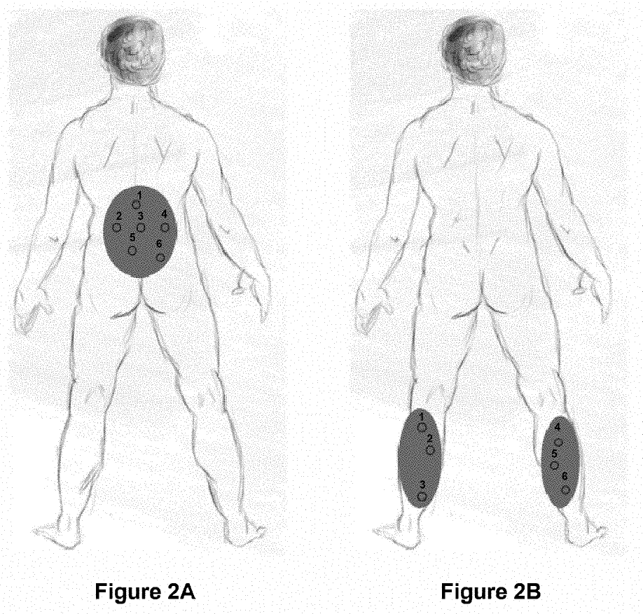

All subjects will receive the same treatments. There will be no subdivision into treatment groups. The test fields will be numbered with 1, 2, 3 etc. As far as possible the following procedure for numbering will be followed: The numbering should begin with the uppermost or most proximal site on the left from the investigator's view. Fields along the same line should be numbered from left to right. If more than one plaque is included, numbers for the fields on the next plaque will be assigned likewise starting with the next free number. The exact location of each test field in relation to the outline of the plaque(s) will be traced on a transparent plastic sheet which will be kept with the subject's source data. FIG. 2 shows representatives of two possible scenarios. The actual distribution of plaques and test fields may vary individually.

Plaque psoriasis on the trunk or extremities located on up to three comparable psoriatic plaque(s) adequate to treat six separate fields (min 1.5 cm apart) will be identified, followed by descaling with a mild detergent. Photographs of the identified treatment site(s) will be obtained. Sonography of the identified treatment sites will occur to ensure that each treatment site has at least a 200 μm EPB thickness of the psoriatic infiltrate.

The subjects will be randomised for allocation of study drug to the 6 test fields (1.1 cm² each). Study drug application will begin on Day 1. Six holes corresponding to the identified fields will be punched in one or multiple hydrocolloid dressing(s) (Varihesive® E). Cotton webril pads will be inserted into each of 6 special aluminium chambers (Duhring chambers). Approximately 200 μL of each study drug will be applied to each cotton webril pad and the Duhring chambers will be immediately seated in holes punched in a hydrocolloid dressing. The chambers will then be fixed in place with Leukosilk®. The chambers will be removed before each new application.

### Global Assessment of Test Field

A clinical assessment (global assessment, compared to surrounding plaque skin) of the test fields will be performed using a 5-point score. For the Baseline Visit all scores will be=0.
−1=worsened
0=unchanged (no effect)
1=slight improvement
2=clear improvement but not completely healed
3=completely healed

### Clinical Assessment of Erythema

A clinical assessment of erythema will be made on the treatment fields using a 5-point scale:
0=none
1=mild (slight pinkness present)
2=moderate (definite redness easily recognised)
3=severe (intense redness)
4=very severe (very intense redness)

### Sonography

Sonographic measurements will be performed using a 22 MHz high frequency sonograph (DUB SkinScanner, Taberna pro Medicum, Lueneburg) equipped with a 2D-head. Serial A-scans will be composed and represented on a monitor as a section of the skin. A lateral resolution of approximately 200 μm and an axial resolution of 80 μm are possible. Dependent on the echo patterns, components of the epidermis, dermis and subcutis are represented. Therefore, exact measurement of skin thickness is possible. The psoriatic inflammatory infiltrate is seen as a clearly definable EPB below the entrance echo. For the analysis, the thickness of the EPB measured in μm, as a surrogate for the psoriatic infiltrate thickness, will be determined and documented using screen shots with visible measuring lines and values. Screen shots of the measurements will be created.

Sonographic measurements will be performed on Days 1 (Baseline), 8, 12 and 19 (EoT).

Chromametry measurement of Erythema

Erythema measurements will be performed with a Chromameter CR-400 (KonicaMinolta).

In accordance with the L*a*b* system, a decrease or increase in the a*-value (colour range from green [−] to red [+]) corresponds to a decrease or increase, respectively, in the degree of erythema. At the same time differences in the chromatic “blue-yellow axis” (b*, tan component) and brightness (L*) will be measured. The L*- and b*-values will not be recorded in this trial. Three measurements will be taken from each test site at each measurement series. The print-outs of the 3 a*-values will be considered source data. Additionally, the a*-value of all three measurements will be recorded in the eCRF.

The measurement of skin redness by chromametry is a standard method in the assessment of skin condition. Similar to sonography, it is generally recognised as reliable and accurate and is widely used to provide standard variables in clinical trials.

### Photography

Photographic documentation of the plaque(s) (overview) will be performed using a digital camera on Day 1 after mild detergent decaling and definition of test fields and prior to application of hydrocolloid dressing.

Further photographic documentation will be performed using a digital camera on Day 19 (EoT) after mild detergent decaling of the test fields. Prior to taking the photograph, the plaques(s) will be marked with a specific label indicating trial number, subject number, day and test field number which will be visible on the photographs. The photographs will be taken under standardised conditions (e.g., distance, illumination).

### Testing

Subjects will return to the clinic daily through Day 19, except on Day 7 and Day 14. The Duhring chambers will be removed each day and the treatment fields gently cleansed with a dry, soft tissue to remove any study drug residues. Study drug will be applied at each visit through Day 18. On Day 7 and 14 the occlusion will remain in place and no new application will be performed (no visit at the site). The hydrocolloid dressing will be replaced on Day 8 and Day 12, or if necessary.

On Day 8 and Day 12, Global assessment of test field will be carried out. The clinical assessment of the extent of erythema and chromametry will be conducted on the treatment fields. These clinical assessments will be done prior to descaling with a mild detergent and sonographic measurements.

Subjects will return to the clinic for their final visit on Day 19. During this visit clinical assessments (global assessment and erythema), and photographs of the treatment fields will be obtained. In addition, chromametry will be performed to measure skin redness (erythema). Descaling of the treatment fields with a mild detergent will occur. Sonography at 22-MHz will be conducted to measure the thickness of the EPB of the psoriatic inflammatory infiltrate. A 4.5 mm biopsy will be obtained from each of the treatment fields and from untreated/uninvolved skin in a subset of 5 subjects. All dressing will be removed and the subject will be discharged from the study. For subjects having biopsies, wound control will take place for approximately 7 to 10 days after taking the biopsy.

### Statistical Methods

### Analysis Sets:

This study will be evaluated using 3 analysis sets. The safety-evaluation-set (SES) will include all subjects who received any study drug at least once; all safety analyses will be based on the SES.

The full-analysis-set (FAS) will include all randomised subjects who received at least one dose of study drug and had at least one post-Baseline assessment. The intention-to-treat (ITT) analysis will be based on the FAS.

The valid-cases-set (VCS), a subset of the FAS, will include all subjects who completed the trial without any protocol deviation that interfered with the efficacy evaluation.

The per-protocol (PP) analysis will be based on the VCS and will be considered as primary analysis.

Demographic and background data will be summarised using descriptive statistical methods. Continuous data will be summarised by mean, standard deviation, median, minimum and maximum. Categorical demographic data will be summarised by frequency tables. Medical history, previous and concomitant therapies will be listed.

### Efficacy Analyses:

Since this is an exploratory, proof-of-concept trial, no formal hypotheses are postulated. The data will be evaluated descriptively.

The change and percent change from Baseline in EPB thickness of the psoriatic inflammatory infiltrate will be determined as the difference of each post-Baseline assessment to the Baseline assessment. Additionally, the AUC of change in the EPB thickness of the psoriatic inflammatory infiltrate will be determined for the assessment period of Baseline to Day 8, 12, and 19 using the linear trapezoidal rule. The change and percent change from Baseline to Day 8, 12 and 19 in erythema (chromametry; a*-values) will also be determined.

The total clinical assessment scores (global and erythema) will be determined as the sum over the assessments for each subject and treatment.

### Primary Efficacy Analysis:

The analysis of the primary efficacy endpoint will comprise pairwise comparisons of the treatments on change from Baseline to Day 19 (EoT) in thickness of the EPB of the psoriatic inflammatory infiltrate, evaluated within a mixed effect model, including treatment, Baseline EPB thickness values and the interaction of treatment and Baseline EPB thickness of the psoriatic inflammatory infiltrate as fixed effects and covariates and a random subject effect.

The estimated differences in least square means (Ismeans) with 95% confidence intervals and the p-values will be reported.

### Secondary Efficacy Analysis:

The percent change from Baseline to Day 8, 12 and 19 (EoT) in thickness of the EPB of the psoriatic inflammatory infiltrate, the change from Baseline to Day 8 and 12 in thickness of the EPB of the psoriatic inflammatory infiltrate, AUC of change from Baseline in the EPB thickness of the psoriatic inflammatory infiltrate to Day 8, 12 and 19, and the change and percent change from Baseline to Day 8, 12 and 19 in erythema (chromametry) will be performed in the same manner as for the primary endpoint.

In addition, descriptive statistics will be provided for thickness of the EPB of the psoriatic inflammatory infiltrate, erythema (chromametry) and for clinical assessment scores, including changes from Baseline in thickness of the EPB of the psoriatic inflammatory infiltrate and erythema (chromametry), and will be presented by treatment and visit.

Descriptive statistics for AUC of change from Baseline in thickness of the EPB of the psoriatic inflammatory infiltrate and for total clinical assessment score (sum of the clinical assessment scores over the assessments times for each subject and treatment) will be presented by treatment.

Frequency counts will be presented by treatment and visit for clinical assessment scores (global and erythema).

Line plots will be presented for mean absolute assessments and for changes from Baseline. Bar plots with confidence intervals will be provided for the AUC of change in thickness of the EPB of the psoriatic inflammatory infiltrate and the total clinical assessment score.

Histology, immunohistochemistry, and gene expression analyses will be summarised comparing outcomes between treatments.

The last observation carried forward (LOCF) principle will be applied to impute missing assessments of efficacy variables (e.g., due to a missed visit or due to treatment discontinuation). No other imputation will be applied.

In general, subjects dropping out of the trial will be included in the safety and the ITT efficacy analyses, but will be excluded from the PP efficacy analysis, as far as they qualify for the analysis populations.

Given a similar effect for a betamethasone valerate ointment, a sample size of 15 will have >90% power to detect a difference in means of 435 μm (e.g., a First condition mean (Day 19), μ1, of 435 and a Second condition mean, (Day 1, Baseline), μ2, of 0, assuming a standard deviation 10% higher than in the former study (1.1*263=289), using a paired t-test with a 0.050 two-sided significance level.

### Example 3

Residual cannabidiol concentrations for a range of compositions were measured before identifying the compositions most suitable for use in the dosage regimens of the present invention, as summarised in Table 4, below.

TABLE 4
Concentration of Cannabidiol (CBD) on skin after
evaporation of volatile solvents

| | | | | Final CBD |
|---|---|---|---|---|
| | | | | Concentration |
| | Initial CBD | Volatile | Residual | After |
| | Concentration | Component(s) | solvent(s) | Evaporation |
| Formulation | % w/w | % w/w | % w/w | % w/w |
| 1 | 0.1 | 99.7 | 0.2 | 33.3 |
| 2 | 0.5 | 99.3 | 0.2 | 71.4 |
| 3 | 1.0 | 98.8 | 0.2 | 83.3 |
| 4 | 1.0 | 98.0 | 1.0 | 50.0 |
| 5 | 5.0 | 94.0 | 1.0 | 83.3 |
| 6 | 10.0 | 89.0 | 1.0 | 90.9 |
| 7 | 1.0 | 97.0 | 2.0 | 33.3 |
| 8 | 5.0 | 93.0 | 2.0 | 71.4 |
| 9 | 10.0 | 88.0 | 2.0 | 83.3 |
| 10 | 1.0 | 96.0 | 3.0 | 25.0 |
| 11 | 5.0 | 92.0 | 3.0 | 60.0 |
| 12 | 10.0 | 87.0 | 3.0 | 76.9 |

TABLE 5
Compositions for use in one or more of the
abovementioned studies (% w/w)

| | BTX | BTX | Vehicle | |
|---|---|---|---|---|
| Ingredients | 1308-1 | 1308-3 | Control | Function |
| Cannabidiol (CBD) | 5.0 | 20.0 | | Active |
| Hexamethyldisiloxane | 91.8 | | 96.8 | Volatile solvent |
| Isopropyl alcohol | | 3.4 | | Volatile Solvent |
| Dimethicone | 1.1 | | 1.1 | Viscosity |
| | | | | modifier |
| Transcutol P | | 62.5 | | Volatile solvent |
| Polypropylene Glycol- | 2.1 | 14.1 | 2.1 | Emollient/ |
| 15 (PPG-15) Stearyl | | | | residual solvent |
| Ether | | | | |
| Total | 100 | 100 | 100 | |

Numerous variations and modifications of the above-described modes of carrying out the various embodiments of this invention will be apparent to those skilled in the art, based on the above teachings related to the disclosed invention, without departing from the basic inventive concepts. The above embodiments of the invention are merely exemplary and should not be construed to be in any way limiting and all such variations and modifications are to be considered within the scope of the present invention, the nature of which is to be determined from the foregoing description.

### REFERENCES

1. Parisi R, Symmons D P, Griffiths C E, Ashcroft D M. Identification and Management of Psoriasis and Associated Comorbidity (IMPACT) project team. Global epidemiology of psoriasis: a systematic review of incidence and prevalence. J Invest Dermatol 2013 February; 133(2): 377-385.
2. Nestle F O, Di Meglio P, et al. Skin immune sentinels in health and disease. Nat Rev Immunol 2009; 9 (10): 679-91.
3. Nestle F O, Kaplan D H, et al. Psoriasis. N Engl J Med 2009; 361(5): 496-509.
4. Mercy K M, Gordon K B, Paller A S. Patient satisfaction and quality of life in psoriasis and psoriatic arthritis. JAMA 2014 Dec. 24-31; 312(24): 2676-2677.
5. Olah A, Toth B, Borbiro I, et al, Cannabidiol exerts sebostatic and anti-inflammatory effects on human sebocytes, J Clin Invest 2014; 124(9); 3713-3724
6. Fernandez-Ruiz J, Sagredo O, Pazos M, et al, Cannabidiol for Neurodegenerative Disorders: Important New Clinical Applications for this Phytocannabinoid? Br J Clin Pharmacol 2012:75(2); 323-333
7. Volkow N, The Biology and Potential Therapeutic Effects of Cannabidiol: “Cannabidiol: Barriers to Research and Potential Medical Benefits” Presentation to NIDA Jun. 24, 2015
8. Dumas K J, Scholtz J R. The psoriasis bio-assay for topical corticosteroid activity. Acta Derm Venereol 1972; 52(1): 43-48.
9. Bangha E, Elsner P. Evaluation of topical antipsoriatic treatment by chromametry, visiometry and 20-MHz ultrasound in the psoriasis plaque test. Skin Pharmacol 1996; 9: 298-306.
10. Snape, S D, Wigger-Alberti, W and Goehring, U M. A Phase I randomized trial to assess the effect on skin infiltrate thickness and tolerability of topical phosphodiesterase inhibitors in the treatment of psoriasis vulgaris using a modified psoriasis plaque test. Br J Dermatol 2016; Epub ahead of print.
11. Gupta A K, Turnbull D H, Harasiewicz K A, Shum D T, Watteel G N, Foster S, Sauder D N. The use of high-frequency ultrasound as a method of assessing the severity of a plaque of psoriasis. Arch Dermatol 1996; 132: 658-662.
12. Kvist P H, Svensson L, Hagberg O, Hoffmann V, Kemp K, Ropke M A. Comparison of the effects of vitamin D products in psoriasis plaque test and a murine psoriasis xenograft model. J Transl Med 2009; 7: 107.
13. Kragballe K, Noerrelund K L, Lui H, Ortonne J P, Wozel G, Uurusuma T, Fleming C, López Estebaranz J L, Hanssen L I, Persson L-M. Efficacy of once-daily treatment regimens with calcipotriol/betamethasone dipropionate ointment and calcipotriol ointment in psoriasis vulgaris. Br J Dermatol 2004; 150: 1167-1173.
14. Kaufmann R, Bibby A J, Bissonnette R, Cambazard F, Chu A C, Decroix J, Douglas W S, Lowson D, Mascaro J M, Murphy G M, Stymme B. A new calcipotriol/betamethasone dipropionate formulation (Daivobet® T M) is an effective once-daily treatment for psoriasis vulgaris. Dermatology 2002; 205: 389-393.
15. Reich K, Brewley A. What is new in topical therapy for psoriasis. JEADV 2011; 25 (Suppl. 4), 15-20.
16. Kozela E, Juknat A, Kaushansky N, Ben-Nun A, Coppola G, Vogel Z. Cannabidiol, a non-psychoactive cannabinoid, leads to EGR2-dependent anergy in activated encephalitogenic T cells. J Neuroinflammation. 2015; 12: 52
17. Tawada C, Kanoh H, Nakamura M, Mizutani Y, Fujisawa T, Banno Y et al. Interferon-γ decreases ceramides with long-chain fatty acids: possible involvement in atopic dermatitis and psoriasis. J. Invest Dermatol. 2014; 134: 712-718.
18. De Filippis D, Esposito G, Cirillo C, Cipriano M, De Winter B Y, Scuderi C et al. Cannabidiol reduces intestinal inflammation through the control of neuroimmune axis. PLoS One. 2011; 6: e28159
19. Giacoppo S, Galuppo M, Pollastro F, Grassi G, Bramanti P, Mazzon E. A new formulation ofcannabidiol in cream shows therapeutic effects in a mouse model of experimental autoimmune encephalomyelitis. Daru. 2015; 23: 48
20. Lee W-S, Erdelyi K, Matyas C, Mukhopadhyay P, Varga Z V, Liaudet L et al. Cannabidiol limits T cell mediated chronic autoimmune myocarditis: implications to autoimmune disorders and organ transplantation. Mol Med. 2016; 22: 136-146

1. A treatment regime for use in the treatment or prevention of psoriasis, said regime comprising the administration of:
a) between 50 mg and 3000 mg of a topical liquid or gel composition comprising between 1% w/w and 15% w/w cannabinoid, wherein the cannabinoid is dissolved in the liquid or gel composition.

2. The treatment regime of claim 1, wherein the topical composition is administered to the skin between 1 and 5 times per day.

3. The treatment regime of claim 1, wherein the topical composition delivers between 20 mg and 400 mg of cannabinoid per administration.

4. The treatment regime of claim 1, wherein the total daily dose applied to the skin is between 20 mg and 2000 mg cannabinoid.

5. The treatment regime of claim 1 wherein:
a) the topical composition comprises 5% w/w, 10% w/w or 20% w/w cannabinoid; and/or
b) the regime delivers 27.5 mg, 37.5 mg or 75 mg of cannabinoid per administration; and/or
c) the regime delivers 27.5 mg, 55 mg, 75 mg or 110 mg of cannabinoid per day.

6. The treatment regime of claim 1, wherein the cannabinoid is delivered in a composition comprising: (i) a volatile solvent; and (ii) a residual solvent that is less volatile than (i).

7. The treatment regime of claim 6, wherein the volatile solvent is chosen from the following: a non-polymeric siloxane, a C2-C6 alcohol and a combination of both.

8. The treatment regime of claim 7, wherein the composition comprises 85-95% w/w siloxane and 1-10% wt/wt C2-C6 alcohol.

9. The treatment regime of claim 7, wherein the siloxane has two or three silicon atoms per molecule.

10. The treatment regime of claim 9, wherein the siloxane is hexamethyldisiloxane.

11. The treatment regime of claim 7, wherein the composition comprises 60-80% wt/wt C2-C6 alcohol.

12. The treatment regime of claim 11, wherein the C2-C6 alcohol is transcutol and/or isopropyl alcohol.

13. The treatment regime of claim 6, wherein the residual solvent is a compound from the list comprising: fatty acids, fatty acid alcohols, fatty alcohols, glycols, alkanes, ethers of any of these, and combinations thereof.

14. The treatment regime of claim 13, wherein the composition comprises 1-10% wt/wt of residual solvent.

15. The treatment regime of claim 14, wherein the residual solvent is a compound from the list comprising: alkyl polypropylene glycol, polyethylene glycol ether, oleyl alcohol, isostearyl alcohol, octyldodecyl alcohol, 2-hexyl decyl alcohol, isohexadecane.

16.-34. (canceled)
